(19) United States
(12) Patent Application Publication
(10) Pub. No.: US 2007/0071754 A1
(43) Pub. Date: Mar. 29, 2007

(54) Method to ameliorate inflammation

(76) Inventor: Gholam A. Peyman, Sun City, AZ (US)

Correspondence Address: WOOD, HERRON & EVANS, LLP, 2700 CAREW TOWER, 441 VINE STREET, CINCINNATI, OH, 45202, US

(21) Appl. No.: 11/234,970

(22) Filed: Sep. 26, 2005

(51) Int. Cl. A61K 39/395; A61K 31/56; A61K 31/47; A61K 31/192; A61K 31/16

(52) U.S. Cl. 424/155.1; 514/171; 514/311; 514/569; 514/570; 514/629

(57) ABSTRACT

Administering to a patient a concentration of at least one anti-inflammatory agent and bevacizumab at a concentration sufficient to reduce fluid leakage from new vessels associated with angiogenesis.

A method of controlling, reducing, or preventing inflammation, an anti-inflammatory response, and/or effects of an anti-inflammatory response. The method provides to a patient the anti-angiogenic agent bevacizumab (Avastin®, Genentech, Inc. South San Francisco Calif.) with one or more anti-inflammatory agent(s). Bevacizumab is administered under conditions sufficient to reduce the growth and proliferation of new blood vessels, which are inherently leaky, and hence reduce fluid leakage from these new vessels into the surrounding tissues. Bevacizumab is administered in conjunction with one or more anti-inflammatory agent(s) known in the art. These include, but are not limited to, steroids, anti-prostaglandins, matrix metalloproteinase inhibitors, non-steroidal anti-inflammatory drugs, etc.

The method supplements anti-inflammatory agents. The method controls inflammation and counteracts the action of angiogenic agents such as vascular endothelial growth factor (VEGF) on the permeability of a vessel wall, thereby reducing or preventing the resulting tissue damage due to fluid leakage from the vessel (extravasation). The method is applicable to any tissue or organ in the body, and to any cause of inflammation such as autoimmune disease, viral and/or bacterial infection. etc. In one embodiment, the method controls, reduces, or prevents tissue damage in the brain. In one embodiment, the method controls, reduces, or prevents tissue damage in the eye.

Inflammation is a localized, protective response of vascularized tissue to sub-lethal tissue injury or destruction. The response functions to destroy, dilute, or sequester both the injurious agent and the injured tissue.

Inflammation can be classified according to duration as either acute or chronic. In the acute form of an inflammatory response, classical signs are pain, heat, redness, swelling, and loss of function. Histologically, there are a complex series of events including dilatation of arterioles, capillaries and venules, with increased permeability and blood flow, exudation of fluids including plasma proteins, and leukocyte migration and accumulation at the site of injury. This reaction may trigger a systemic response such as fever, leukocytosis, protein catabolism, and altered hepatic synthesis of plasma proteins such as C-reactive protein. Chronic inflammation is characterized by macrophage and lymphocyte infiltration into the affected and surrounding tissue.

Inflammation is a homeostatic response to destroy or inactivate invading pathogens. In cases of autoimmune diseases such as rheumatoid arthritis, etc., inflammation is a response against self. The inflammatory process removes waste and debris and restores normal function, either through resolution or repair. Tissue structure is normal after resolution, whereas repair leads to a functional, but morphologically altered, organ. In acute inflammation, tissue damage is followed by resolution, whereas in chronic inflammation, damage and repair continue concurrently. The initial inflammatory response is usually acute, and may or may not evolve into chronic inflammation. However, chronic inflammation is not always preceded by an acute phase. Although usually beneficial to the organism, inflammation itself may lead to tissue damage, resulting in escalation of chronic inflammation. Inflammation underlies the pathology of virtually all rheumatologic diseases. The severity of disorders, such as arthritis, is classified according to the degree of inflammation and its destructive effects.

Angiogenesis is the growth of new blood vessels from pre-existing vasculature. It is a fundamental process required for embryogenesis, growth, tissue repair after injury, and the female reproductive cycle. It also contributes to the pathology of conditions such as cancer, age related macular degeneration, psoriasis, diabetic retinopathy, and chronic inflammatory diseases in joints or lungs. Angiogenesis is stimulated when hypoxic, diseased, or injured tissues produce and release angiogenic promoters such as vascular endothelial growth factor (VEGF) or fibroblast growth factor (FGF)-1. These angiogenic factors stimulate the migration and proliferation of endothelial cells in existing vessels and, subsequently, the formation of capillary tubes and the recruitment of other cell types to generate and stabilize new blood vessels.

Angiogenic factors may be pro-inflammatory factors. Agents that inhibit angiogenesis, such as TNP470, integrin av antagonists, 2-methoxyestradiol, paclitaxel, and P38 mitogen activated protein kinase inhibitors, may inhibit synovitis. Expression of adhesion molecules such as integrin avb3 and e-selectin are upregulated in new vessels, and new vessels appear sensitive to inflammogens. The angiogenic factor FGF-1 enhances antigen-induced synovitis in rabbits, but is not pro-inflammatory when administered alone. However, angiogenesis occurs in the absence of inflammation such as during embryonic growth and in the female reproductive cycle. Thus, inflammation and angiogenesis can occur independently. Coexistence of inflammation and angiogenesis may lead to more severe, damaging, and persistent inflammation.

Angiogenesis enhances tumor growth, and anti-angiogenic agents are used clinically. Mechanisms by which new vessels enhance tumor growth include providing metabolic requirements of the tumor, generating growth factors by vascular cells, and inhibiting apoptosis. Inhibiting the function of growth factors such as VEGF can reduce or prevent pathological angiogenesis in tumors.

Angiogenesis may also contribute to thickening of airways in asthma and of lung parenchyma in pulmonary fibrosis, and to growth of sarcoid granulomas. Growth of granulation tissue into airspaces also may be angiogenesis-dependent in bronchi after lung transplant and in alveoli after acute lung injury or in other forms of pulmonary fibrosis. Angiogenesis may also contribute to growth of the synovial pannus in rheumatoid arthritis. Interposition of expanded, innervated synovium between articulating surfaces may contribute to pain on movement. In each of these situations, the expanded tissue may impair function.

The new blood vessels that result from angiogenesis have incomplete walls and are particularly susceptible to disruption and fluid extravasation. This has been proposed as a cause of pulmonary hemorrhage in inflammatory lung disease. Hemosiderin deposits and extravasated erythrocytes are commonly present in inflammatory synovitis, although the contribution of angiogenesis to synovial microhemorrhage is unknown, and its contribution to synovial inflammation remains unclear. The inflammatory potential is evident, however, in patients with hemophilia.

Angiogenesis occurs as an orderly series of events, beginning with production and release of angiogenic growth factors (proteins) that diffuse into nearby tissues. The angiogenic growth factors bind to specific receptors located on the endothelial cells of nearby preexisting blood vessels. Once growth factors bind to their receptors, the endothelial cells are activated and begin to produce enzymes and other molecules that dissolve tiny holes in the sheath-like basement membrane that surrounds existing blood vessels. The endothelial cells begin to divide and proliferate, and they migrate through the holes of the existing vessel towards the diseased tissue or tumor. Specialized adhesion molecules or integrins (avb3, avb5) help to pull the new blood vessels forward. Additional enzymes, termed matrix metalloproteinases (MMP), are produced and dissolve the tissue in front of the sprouting vessel tip in order to accommodate it. As the vessel extends, the tissue is remolded around the vessel. Sprouting endothelial cells roll up to form a blood vessel tube and individual blood vessel tubes connect to form blood vessel loops that can circulate blood. The newly formed blood vessel tubes are stabilized by smooth muscle cells, pericytes, fibroblasts, and glial cells that provide structural support, permitting blood flow to begin.

VEGF is a specific angiogenesis growth factor that binds to receptors on blood vessels and stimulates the formation of new blood vessels. VEGF is a potent inducer of both endothelial cell proliferation and migration, and its biologic activities are largely specific for endothelial and vascular smooth muscle cells. Unlike basic fibroblast growth factor (bFGF), high levels of VEGF are not present in early surgical wounds. Rather, VEGF levels peak seven days after the wound is created, at which point VEGF appears to be a major stimulus for sustained induction of blood vessel growth and high levels of PDGF have been shown. There are abundant sources of VEGF in wounds. Many cell types produce VEGF, including keratinocytes, macrophages, fibroblasts, and endothelial cells. Thus, there is massive VEGF secretion, particularly in the setting of hypoxia, which is often observed in wounds.

Bevacizumab (rhuMab VEGF; Avastin®, Genentech, Inc., South San Francisco, Calif.) inhibits the action of VEGF. Bevacizumab is a recombinant humanized monoclonal IgG1 antibody that binds to and inhibits the biologic activity of human VEGF in in vitro and in vivo assay systems by preventing binding of VEGF with its receptor on the surface of vascular endothelial cells, thus preventing endothelial cell proliferation and new vessel formation. Bevacizumab contains human framework regions and the complementarity-determining regions of a murine antibody that binds to VEGF; it has a molecular weight of about 149 kilodaltons. Bevacizumab, by binding to VEGF, blocks VEGF from binding to receptors and thus blocks angiogenesis. Bevacizumab is typically administered by intravenous infusion, diluted in 0.9% sodium chloride for injection from a 25 mg/ml preparation.

Among the available anti-inflammatory agents, many have a target of action to block or ameliorate the actions of pro-inflammatory signals, such as histamine and cytokines. Although this provides some relief from the harmful effects of inflammation, it does not address the cause of the problem. Leukocytes and macrophages, which release pro-inflammatory factors into affected areas, are allowed access to the inflamed tissue following new blood vessel formation.

The inventive method administers an anti-inflammatory agent simultaneously or concomitantly with bevacizumab and thus controls, reduces or prevents an inflammatory response. Other anti-VEGF compounds such as Lucentis® or Macugen® may be included. The method may be used for any tissue including, but not limited to, eye, lung, bone, brain, and muscle. The method may be used on patients at risk for developing inflammation. The method may be used on patients with inflammation and/or inflammatory process from any cause, including but not limited to autoimmune diseases, diseases with an immune component, ischemic diseases, infectious diseases, allergen-induced inflammation, and other degenerative diseases.

An effective amount of an anti-inflammatory agent is administered to a patient at a standard dose known to one skilled in the art. As one example, prednisone is administered for a systemic dose in the range between about 5 mg to about 100 mg daily. As another example, Solu-medrol® is administered intravenously in a single dose of about 1 mg. Other anti-inflammatory agents, possible routes of administration, doses, etc. are know to one skilled in the art. The agent may be administered by any route including enteral and parenteral route, for example, intravenously, orally, ocularly, etc. One skilled in the art will appreciate that the route of administration may vary due to factors such as agent solubility, patient needs, dose required, etc. The anti-inflammatory agent may be a fast-acting anti-inflammatory agent, a slow acting anti-inflammatory agent, or both a fast-acting and a slow-acting anti-inflammatory agent. The anti-inflammatory agent may be formulated for delayed and/or extended release to provide effects over a longer period of time.

Examples of anti-inflammatory agents include, but are not limited to, the following: colchicine; a steroid such as triamcinolone (Aristocort®; Kenalog®), anacortave acetate (Alcon), betamethasone (Celestone®), budesonide Cortisone, dexamethasone (Decadron-LA®; Decadron® phosphate; Maxidex® and Tobradex® (Alcon)), hydrocortisone methylprednisolone (Depo-Medrol®, Solu-Medrol®), prednisolone (prednisolone acetate, e.g., Pred Forte® (Allergan), Econopred and Econopred Plus® (Alcon), AK-Tate® (Akorn), Pred. Mild® (Allergan), prednisone sodium phosphate (Inflamase Mild and Inflamase Forte® (Ciba), Metreton® (Schering), AK-Pred® (Akorn)), fluorometholone (fluorometholone acetate (Flarex® (Alcon), Eflone®), fluorometholone alcohol (FML® and FML-Mild®, (Allergan), Fluor OP®), rimexolone (Vexol® (Alcon)), medrysone alcohol (HMS® (Allergan)), lotoprednol etabonate (Lotemax® and Alrex® (Bausch & Lomb), and 11-desoxcortisol; an anti-prostaglandin such as indomethacin; ketorolac tromethamine; ((±)-5-benzoyl-2,3-dihydro-1H-pyrrolizine-1-carboxylic acid, a compound with 2-amino-2-(hydroxymethyl)-1,3-propanediol (1:1) (ACULAR® Allegan), OCUFEN® (flurbiprofen sodium 0.03%), meclofenamate, flurbiprofen, and the pyrrolo-pyrrole group of non-steroidal anti-inflammatory drugs; a macrolide such as sirolimus (rapamycin), pimocrolous, tacrolimus (FK506), cyclosporine (Arrestase), everolimus 40-O-(2-hydroxymethylenrapamycin), ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, lincosamide, biolimus, ABT-578 (methylrapamycin), and derivatives of rapamycin such as temsirolimus (CCI-779, Wyeth) and AP23573 (Ariad); a non-steroidal anti-inflammatory drug such as derivatives of acetic acid (e.g. diclofenac and ketorolac (Toradol®, Voltaren®, Voltaren-XR®, Cataflam®)), salicylate (e.g., aspirin, Ecotrin®), proprionic acid (e.g., ibuprofen (Advil®, Motrin®, Medipren®, Nuprin®)), acetaminophen (Tylenol®), aniline (e.g., aminophenolacetaminophen, pyrazole (e.g., phenylbutazone), N-arylanthranilic acid (fenamates) (e.g., meclofenamate), indole (e.g., indomethacin (Indocin®, Indocin-SR®)), oxicam (e.g., piroxicam (Feldene®)), pyrrol-pyrrole group (e.g., Acular®), antiplatelet medications, choline magnesium salicylate (Trilisate®), cox-2 inhibitors (meloxicam (Mobic®)), diflunisal (Dolobid®), etodolac (Lodine®), fenoprofen (Nalfon®), flurbiprofen (Ansaid®), ketoprofen (Orudis®, Oruvail®), meclofenamate (Meclomen®), nabumetone (Relafen®), naproxen (Naprosyn®, Naprelan®, Anaprox®, Aleve®), oxaprozin (Daypro®), phenylbutazone (Butazolidine®), salsalate (Disalcid®, Salflex®), tolmetin (Tolectin®), valdecoxib (Bextra®), sulindac (Clinoril®), and flurbiprofin sodium (Ocufen®), an MMP inhibitor such as doxycycline, TIMP-1, TIMP-2, TIMP-3, TIMP-4; MMP1, MMP2, MMP3, Batimastat (BB-94), TAPI-2,10-phenanthroline, and marimastat. The composition may contain other anti-VEGF compounds such as ranibizumab (Lucentis®, Genentech) and/or pegaptanib (Macugen®). The composition may contain anti-PDGF compound(s) such as imatinib mesylate (Gleevec®) and/or anti-leukotriene(s) such as genleuton, montelukast, cinalukast, zafirlukast, pranlukast, zileuton, BAYX1005, LY171883, and MK-571 to account for the involvement of factors besides VEGF in neovascularization. The composition may additionally contain other agents including, but not limited to, transforming growth factor β (TGFβ), interleukin-10 (IL-10), aspirin, a vitamin, and/or an antineoplastic agent.

An effective amount of bevacizumab, and optionally one or more other agents as previously described, is administered with the anti-inflammatory agent(s). Administration of either agent may be by any route, and the agents may be administered by the same route or by different routes, including enteral, parental, and ocular routes such as intravitreal injection, subconjunctival injection, retrobulbar injection, topical, etc. In one embodiment, the administered dose of bevacizumab is less than about 5 mg/0.1 ml. In another embodiment, the administered dose of bevacizumab ranges from 0.1 mg/ml to about 50 mg/ml. In another embodiment, the dose of bevacizumab administered systemically ranges from about 0.05 mg/ml to about 5 mg/ml. In one embodiment, the dose of bevacizumab administered intraocularly (e.g., intravitreally) is about 0.05 mg/ml to about 5 mg/ml. In one embodiment, the dose of bevacizumab administered topically to the eye is up to 5 mg/ml, and in another embodiment it may be higher. In one embodiment, the administered dose of anti-inflammatory agent ranges from about 0.01%^{w/w} to about 10%^{w/w}. In another embodiment, the administered does of anti-inflammatory agent ranges from about 0.05 mg/ml to about 100 mg/ml. The therapies may be administered in any sequence, that is, bevacizumab may be administered before or after the anti-inflammatory agent(s), or they may be administered essentially simultaneously. In embodiments, the time between the administration of bevacizumab and the anti-inflammatory agent(s) may be within a few minutes, within a few hours, within a few days, or up to about 45 days.

Without being limited by a specific theory, the inventive method of administering an anti-inflammatory agent and bevacizumab may provide a synergistic effect. The inventive method alleviates the response to pro-inflammatory factors, and also reduces the recurrence and slows the progression of additional new vessels, thereby diminishing the presence of pro-inflammatory secreting cells.

Either a fast-acting agent anti-inflammatory agent or an anti-inflammatory agent of sustained duration may be used, as known to one skilled in the art. A fast-acting agent is one that exerts a therapeutic effect in a relatively short period, due either to its formulation, its chemistry, or both. is an example of a fast-acting anti-inflammatory agent. An agent that will provide sustained therapy over a prolonged period may occur due to the agent's formulation as a controlled-release or delayed release substance, its chemistry, or both. Examples of suitable longer-acting anti-inflammatory agents include macrolides.

Solutions may be prepared using a physiological saline solution as a vehicle. The pH of the ophthalmic solutions may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

The formulations may also contain pharmaceutically acceptable excipients known to one skilled in the art such as preservatives, stabilizers, surfactants, chelating agents, antioxidants such a vitamin C, etc. Preservatives include, but are not limited to, benzalkonium chloride, chlorobutanol, thimerosal, phenylmercuric acetate and phenylmercuric nitrate. A surfactant may be Tween 80. Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylated hydroxyanisole, butylated hydroxytoluene, etc.

In various embodiments, the compositions may contain other agents. The indications, effective doses, formulations, contraindications, vendors, etc. of these are available or are known to one skilled in the art.

The concentration of anti-inflammatory agent used in a particular embodiment may depend upon the particular class of agent (e.g., steroid, anti-prostaglandin, etc.), and/or particular agent (e.g., a lipophilic compound versus a water-soluble compound), and/or its formulation (e.g., extended release, delayed release, etc.), and/or its route of administration (e.g., intraocular injection versus systemic administration), and/or patient specific variables (e.g., fast or slow metabolizer, age, gender) etc. as will be appreciated by one skilled in the art. Thus, the following concentrations are general ranges only. In embodiments using an anti-inflammatory steroid, the steroid concentration administered may range from about 0.1 mg/ml to about 40 mg/ml; for intraocular administration the steroid concentration may range from about 10 μg/ml to about 400 μg/ml. In embodiments using an anti-prostaglandin, also termed a prostaglandin antagonist, the concentration administered may range from about 0.003%^{w/w} to about 0.3%^{w/w}, with some embodiments having the concentration administered up to about 3%^{w/w}, and some embodiments having the concentration administered up to about 10%^{w/w}.

Acute processes may occur in patients with chronic inflammation. Thus, a regimen to treat chronic inflammation should also include a fast-acting anti-inflammatory component. In treating either acute or chronic inflammation, a slow acting agent may be included to prevent reactivation of the inflammatory process and/or the underlying pathology. Doses may be lower than or the same as those for standard anti-inflammatory treatment. It will be appreciated that the agents include pharmaceutically acceptable salts and derivatives.

In the inventive method, both anti-inflammatory agent and bevacizumab are administered, but their administration is not restricted to a particular sequence. In one embodiment, anti-inflammatory agent is administered essentially simultaneously with or immediately after bevacizumab is administered. In another embodiment, the anti-inflammatory agent is administered and bevacizumab is administered in the same treatment session, within a time frame of a few hours. In another embodiment, the anti-inflammatory agent is administered and bevacizumab is administered after an interval from about one day up to about 45 days. In another embodiment, bevacizumab is administered essentially simultaneously with or immediately thereafter anti-inflammatory agent is administered. In another embodiment, bevacizumab is administered and anti-inflammatory agent is administered in the same treatment session, within a time frame of a few hours. In another embodiment, bevacizumab is administered and anti-inflammatory agent is administered after an interval from about one day up to about 45 days.

Administration of an anti-inflammatory agent and bevacizumab, and optionally other agents such as an anti-PDGF compound, an anti-VEGF compound, etc., may supplement or replace photodynamic therapy (PDT) and hence avoid the retinal damage frequently associated with PDT. PDT is frequently used to reduce or prevent damage from leaky vessels associated with age related macular degeneration and other diseases. A series of PDT treatments is often performed with a cumulative effect that, over time, results in retinal damage that in some cases may be severe. The present invention may obviate the need for PDT thus eliminating associated damage.

It is reported that bevacizumab at a dose of 1 mg was administered as a single intravitreal injection to a patient with neovascular age-related macular degeneration. Rosenfeld et al. Ophthalmic Surg Lasers Imaging 2005; 36:331 which is expressly incorporated by reference herein in its entirety. There was resolution of subretinal fluid after one week, with improved macular appearance maintained for at least four weeks, and no observed inflammation. Thus, bevacizumab at a dose of 5 mg/0.1 ml would not be expected to be toxic.

The invention will be further appreciated with reference to the following example.

Forty eyes belonging to forty male Long Evans pigmented rats (200 g to 250 g) are divided into three groups. Treated eyes are topically administered bevacizumab and an anti-inflammatory agent. One eye of each animal serves as a treated eye and the other eye serves as a non-treated control eye. Artificial corneal burns are induced. All the eyes are examined to exclude any eyes with corneal scars and/or neovascularizations prior to induction. More specifically, topical administration of the described agents are administered twice a day to rats in which corneal burns are artificially induced by application of silver nitrate (70%) and potassium nitrate (30%).

Neovascularization is induced in eyes using silver nitrate cauterization. The animals are first anesthetized by intraperitoneal injection of a mixture of ketamine hydrochloride (25 mg/kg) with xylazine hydrochloride (5 mg/kg). The cornea is then anesthetized by a drop of 0.5% proparacaine and allowed to dry. One cornea of each animal is cauterized by pressing an applicator stick (diameter of 1.8 mm) coated with 75% silver nitrate/25% potassium nitrate (Arzol Chemical Co., Keen NH) to the central cornea for ten seconds (using a stopwatch) under the operating microscope. Excess silver nitrate is removed by rinsing the eyes with balanced salt solution. To increase the reproducibility of the injuries, one investigator cauterizes all animals.

Following cauterization, the animals are randomly divided into four groups to eliminate any potential bias in the degree of burns within the different groups. Group 1 (number of animals (n)=10) receives a topical balanced salt solution. Group 2 (n=10) receives topical bevacizumab (5 mg/ml). Group 3 (n=10) receives topical triamcinolone (10 μg/m). Group 4 (n=10) receives a combination of bevacizumab (5 mg/ml) and triamincolone (10 μg/ml). Two drops of each drug are applied topically to each cornea immediately following cauterization; treatments are administered two times per day for seven days.

The presence of new vessels (neovascularization) and the extent of new vessel formation is assessed by slit lamp photography and histology. Inhibition of vessel proliferation is evaluated by measuring vessel progression from the outer cornea (corneal limbus) into the cornea. It will be appreciated that any reduction of new vessel proliferation and/or regression of existing vessels is therapeutic, and that complete inhibition and/or regression is not required, and also that reduction includes regression of existing vessels.

All animals are anesthetized as described above and their corneas evaluated by slit-lamp microscopy on third and sixth days. Corneal photographs are taken with x25 magnification using a camera attached to the slit-lamp microscope (Topcon SL-7E, Tokyo Japan) on the seventh day. Neovascularization is evaluated by an examiner who is blinded as to the treatment groups to minimize the observer bias.

The animals are euthanized in a carbon dioxide chamber under deep general anesthesia. The eyes are enucleated and fixed in 10% formaldehyde. After fixation for 24 hours, the eyes are removed from the fixative and corneas are dehydrated and sectioned. The corneas are then soaked in xylene and paraffin, later they are embedded in paraffin and cut at 1 μm for staining with hematoxylin-eosin (H&E) for light microscopy.

Corneal neovascularization is assessed by scanning (Cano scan 9900F, Canon, Tokyo Japan) the slit lamp photographs into high resolution digital images. The percentage area of corneal neovascularization is determined by outlining the areas with corneal vessels and comparing these to the total corneal surface using image j software (Wayne Rasband at the Research Services Branch, National Institute of Mental Health, Bethesda Md.). The percentage area of the cornea covered by the corneal scar in each eye is also determined. A drawing of corneal blood vessels is made to compare with digital photos and ensure that no vascular area is omitted during calculation of percent area.

For each eye, the extent of burn stimulus response is scored as 0 (no blister, not raised above corneal surface), +1 (small blister, raised slightly above the surface), +2 (medium blister, raised moderately above the surface), or +3 (large blister). Only corneas with a burn stimulus score of +2 or higher are included for the calculation of the mean burn stimulus and neovascularization scores in each group. All photographs are converted to high-resolution digital forms by scanner (Canon scan 9900F, Canon, Tokyo Japan). The corneal surface covered with neovascular vessels is measured on the photographs as the percentage of the total area of the cornea. Image analysis is performed on each cornea using an image processing and analysis software program (Image J 1.31v. Wayne Rasband at the Research Services Branch, National Institute of Mental Health, Bethesda Md.). The area of neovascularization is measured in terms of pixels and its ratio to the entire corneal area was determined as the percentage of corneal neovascularization. A drawing of corneal blood vessels is made for comparison with digital photographs to ensure that no vascular area was missed in the calculation of percent area. The extent of the scar is also evaluated by calculating the percentage of the corneal surface that is covered by the scar.

Percent inhibition is calculated by comparing the mean percentage of neovascularization in each treated group to that in the control group. After scoring the burn stimulus and the percentage of neovascularization for all groups, the animals are sacrificed on the seventh day.

Statistical analyses are performed using each animal as an experimental unit with Statistical Analysis System (SPSS 11.5) software. Kruscal-Vallis and Mann-Whitney U Analysis is conducted and treatment means are separated at p<0.05 with least significant difference (LSD) test. A p value <0.05 is considered significant.

For histopathologic evaluation, sedated animals are euthanized with inhaled CO₂ and enucleation is performed immediately. The globes are penetrated with a 27-gauge needle, 1.0 mm from the limbus at the 3 and 9 o'clock meridians to allow the fixative to fill the eyes rapidly. The eyes are prepared for histologic examination using 10% formaldehyde. After fixation for twenty-four hours, the eyes are removed from the fixative and corneas are dehydrated and sectioned. The corneas are then soaked in xylene and paraffin, and are later embedded in paraffin and cut at 1 μm for staining with hematoxylin and eosin (H&E) for light microscopy.

Light microscopic examination is performed on every microscopic section. Sections are examined by dividing the corneas into two halves through the center of the lesion and are evaluated with regard to the intensity of new vessels, polymorphonuclear (PMN) leucocytes, edema, and fibroblastic activity.

In Group 2, the degree of corneal neovascularization is reduced when bevacizumab administration is compared to control administration, p=0.05. In Group 3, the degree of corneal neovascularization is reduced when triamcinolone administration is compared to control administration, p=0.05. In Group 4, the degree of corneal neovascularization is significantly reduced when the combination of bevacizumab and triamcinolone is compared to control administration, p=0.02. The two compounds at the lowest concentration achieve a synergistic result.

It should be understood that the embodiments of the present invention shown and described in the specification are only preferred embodiments of the inventor who is skilled in the art and are not limiting in any way. For example, the inventive method may be used to treat cerebral edema associated with meningitis by intravenously administering bevacizumab. Therefore, various changes, modifications or alterations to these embodiments may be made or resorted to without departing from the spirit of the invention and the scope of the following claims.

What is claimed is:

1. A method for ameliorating an ocular inflammatory process in a patient comprising providing to the patient a therapeutic concentration of at least one anti-inflammatory agent and bevacizumab at a concentration sufficient to reduce ocular angiogenesis and inflammation and their sequelae.

2. The method of claim 1 further comprising providing another anti-VEGF compound, an anti-PDGF compound, an anti-leukotriene, or combinations thereof.

3. The method of claim 1 wherein bevacizumab is at a concentration up to about 5 mg/0.1 ml.

4. The method of claim 1 where bevacizumab is administered topically to an eye at a concentration up to about 5 mg/ml.

5. (canceled)

6. The method of claim 1 where bevacizumab is administered intraocularly at a concentration ranging from about 0.05 mg/ml to about 5 mg/ml.

7. The method of claim 1 wherein the anti-inflammatory agent is selected from at least one of colchicine, a steroid, a matrix metalloproteinase inhibitor, or a macrolide.

8. (canceled)

9. A method for ameliorating an ocular inflammatory process in a patient comprising providing to the patient a therapeutic concentration of at least one anti-inflammatory agent and bevacizumab at a concentration sufficient to reduce ocular angiogenesis.

10. The method of claim 9 wherein bevacizumab is administered topically, systemically, or by intraocular injection.

11-14. (canceled)
